(19) United States
(12) Patent Application Publication
(10) Pub. No.: US 2024/0428600 A1
(43) Pub. Date: Dec. 26, 2024

(54) TRANSFECTION IMAGING FOR CELL WALL ANNOTATIONS

(71) Applicant: Carl Zeiss Microscopy GmbH, Jena (DE)

(72) Inventors: Manuel AMTHOR, Jena (DE); Daniel HAASE, Jena (DE)

(73) Assignee: Carl Zeiss Microscopy GmbH, Jena (DE)

(21) Appl. No.: 18/747,688

(22) Filed: Jun. 19, 2024

(30) Foreign Application Priority Data
Jun. 20, 2023 (DE) 102023116100.8

(51) Int. Cl. G06V 20/69; G06T 7/00; G06T 7/13; G06V 20/70; G16H 30/40

(52) U.S. Cl. CPC G06V 20/695; G06T 7/0012; G06T 7/13; G06V 20/70; G16H 30/40; G06T 2207/10056; G06T 2207/10064; G06T 2207/30024

(57) ABSTRACT

Various aspects of the disclosure relate to techniques for recognizing cell structures in microscope images. In particular, techniques for recognizing cell walls, i.e. cell edges, in microscope images, e.g. phase contrast images, are described. For this purpose, a machine-learned algorithm, e.g. an artificial neural network, can be used. Techniques of how annotations can be created as a ground truth for the training of the machine-learned algorithm, e.g. based on the fluorescence channel of transfection image data, are described. Further actions can then be performed based on correspondingly localized cell walls, e.g. cell-instance annotations that segment cell instances, for the training of a further machine-learned algorithm.

### FIELD OF THE INVENTION

Various examples of the disclosure relate to techniques related to the recognition of cellular structures in microscope images. Various examples of the invention relate in particular to techniques of machine learning and the training of machine-learned algorithms.

### BACKGROUND OF THE INVENTION

In the fields of biology and medicine, microscopy is used to study biological samples. Microscope image analysis is often used to identify and classify individual cells, for example to gain relevant information about their structure, function, and potential abnormalities. The concept of instance segmentation is often used for this task.

Instance segmentation is the name for a process in which each cell in a microscope image is individually identified and separated from other cells. This means that not only the presence and position of the cells within the image are determined, but also their exact shape and size. Instance segmentation makes it possible to examine cellular properties and behaviors at an individual level.

The challenge of instance segmentation is to identify the boundaries of each cell, especially in complex or densely populated images, in which the cells often overlap or have very similar features. In addition, cells vary in shape, size, and structure, making instance segmentation even more difficult.

To overcome these challenges, various techniques and algorithms are used, including machine-learned algorithms.

Ronneberger et al. have developed a neural network called U-Net, which is specifically designed for segmenting biomedical images. See Ronneberger, O., Fischer, P., & Brox, T. (2015, October). U-net: Convolutional networks for biomedical image segmentation. In International Conference on Medical Image Computing and Computer-assisted Intervention (pp. 234-241). Springer, Cham. U-Net uses a special architecture that allows the network to utilize both local and global context information to make accurate segmentation decisions. The U-net was also used for instance segmentation of cells, see for example Falk, Thorsten, et al. “U-Net: deep learning for cell counting, detection, and morphometry.” Nature methods 16.1 (2019): 67-70.

In order to train machine-learned algorithms, such as the U-net by Ronneberger et al., annotations are used as ground truth (“ground-truth annotations” or simply annotations). These annotations serve as a comparison standard or “source of truth” for the training and/or validation of the machine-learned algorithms (ML algorithms). They provide the correct answers that the ML algorithm should learn to reproduce.

However, one challenge lies in the creation of annotations. The manual creation of annotations requires considerable effort, as each individual cell in the microscope images must be precisely delimited. This is a time-consuming and error-prone task that requires expert knowledge and is therefore often expensive. In addition, differences in interpretation among different annotators can lead to inconsistencies in the annotations, which can affect the quality of the trained model.

### SUMMARY OF THE INVENTION

Manual creation of annotations is therefore a major obstacle to scaling and efficiency of instance segmentation of cells in microscope images. There is a need for solutions that can automate or at least simplify this process to increase efficiency while ensuring the accuracy and reliability of the results.

More generally, there is a need for techniques to train machine-learned algorithms that recognize cell features or cell structures. There is a need to obtain annotations as ground truth for such training.

A computer-implemented method is disclosed. An ML algorithm is trained based on cell wall annotations as the ground truth and a non-fluorescence channel as input.

There are several variants for creating the cell wall annotations. In some variants, the cell wall annotations can be created manually. For example, the cell wall annotations can be manually generated in a phase contrast channel from microscope image data. Alternatively or additionally, it would be possible that the cell wall annotations are generated in a non-fluorescence channel from transfection image data. For this purpose, automated image processing of a fluorescence channel can be performed to localize in this way boundary regions between fluorescence regions and non-fluorescence regions.

The ML algorithm which was trained based on the cell wall annotations can then be used further in various ways. For example, an output of the ML algorithm can be evaluated in order to carry out a further evaluation of a corresponding microscope image based on the cell walls recognized by the ML algorithm. For example, an instance segmentation of cells could be performed based on the recognized cell walls. It would be possible, alternatively or additionally, to count the visible cells and/or to determine a degree of confluence.

Another way of using the ML algorithm which was trained based on the cell wall annotations is to generate further annotations as the ground truth for training a further ML algorithm. For example, cells could be segmented based on the cell wall annotations which are output by the ML algorithm, thus creating cell instance annotations. These cell instance annotations serve for training a further ML algorithm that solves a corresponding segmentation task.

Such techniques described above are based on the finding that it is often easier to obtain cell wall annotations than, for example, to obtain cell instance annotations. Cell wall annotations can be easily created manually in some examples. It is less time consuming to annotate a single cell wall than to write down an entire cell. In a further example it was found that transfection imaging is particularly suitable for automatically determining cell wall annotations on corresponding image data.

### BRIEF DESCRIPTION OF THE DRAWINGS

FIG. 1 schematically illustrates a data processing device according to various examples.

FIG. 2 is a flowchart of one exemplary method.

FIG. 3 is a flowchart of one exemplary method.

FIG. 4 shows an example of data processing of transfection image data.

### DETAILED DESCRIPTION OF THE INVENTION

Techniques for recognizing and localizing structures of cells in microscope images are described below. For example, cell walls or other cell structures can be recognized and localized. It would also be conceivable to recognize cell organelles. Appropriate segmentation of such parts of a cell can be provided. Other localization techniques (e.g. point localization or bounding box) would also be conceivable.

The cell wall is also sometimes referred to as the cell membrane. It surrounds the cell and regulates the entry and exit of substances. It consists mainly of a double layer of phospholipids and proteins. The cell wall is different from other cell components such as cytoplasm, cell nucleus, etc.

In the following text, reference is made in particular to variants in which cell walls are recognized and localized. However, corresponding techniques can also be used in connection with the recognition and localization of other structures in cells.

Techniques that recognize and localize cell walls of cells by means of ML algorithms are used. In particular, deep neural networks can be used, for example convolutional neural networks. Transformer networks can also be used.

Techniques that allow ground-truth annotations to be automatically provided to train an ML algorithm to solve a corresponding task of localizing cell walls are described.

The output of the trained ML algorithm is then a localization of cell walls. The cell walls can be marked, for example, by means of a spline line. A binary mask image that determines the position of cell walls can be output.

This output can be used in several ways. It is possible to carry out further evaluations in a downstream image processing step based on the recognized cell walls. For example, an instance segmentation of cells based on the recognized cell walls (or other recognized structures) can be performed. A degree of confluence could also be determined. Alternatively or additionally, it would be possible to determine the number of cells or the cell density. Such data can then in turn be used as ground-truth annotations for the training of a further ML algorithm, which solves the corresponding tasks (for example cell instance segmentation or determination of the degree of confluence or cells of cells) directly (i.e. without first localizing cell walls). This means that the training of the ML algorithm for the localization of cell walls in such an example is only a means for the purpose of creating further annotations for the training of a further ML algorithm, the task of which is related to or based on the localization of cell walls, but nevertheless has a different focus. Such techniques are based on the finding that the training of an ML algorithm for the localization of cell walls can be carried out comparatively simply and reliably and in particular can also be carried out automatically. Then, based on such a first step, the more complex problem of creating annotations for more complex tasks can be solved in a simplified manner.

To create the annotations that serve as the ground truth for training the ML algorithm that localizes cell walls (this ML algorithm is called the cell wall ML algorithm), transfection imaging is used in some examples.

Transfection imaging is a technique known from the field of molecular biology. There it is used to study the effects of specific genes in living cells. This is achieved by introducing DNA (or RNA) into the cells which encodes the gene of interest, often coupled with a reporter gene which encodes for a fluorescent protein. The result is that cells that have successfully absorbed and integrated the DNA into their genome (successful transfection) express the fluorescent protein and luminesce under the microscope, while cells in which the transfection has failed do not. The microscopy used in such studies usually uses two channels. The first is a phase contrast channel that does not use fluorescence. Phase contrast microscopy is a technique that uses the refraction of light to visualize details in a sample. It allows living cells to be seen in culture without staining or fixation, which is often necessary in fluorescence microscopy. The phase contrast channel thus provides a “natural” view of the cells and can be used to determine their morphology and position. In the techniques described herein, the phase contrast channel is used as input into the machine-learned algorithm. The second channel is the fluorescence channel. In this channel, the fluorescence produced by the expression of the fluorescent protein is detected. Fluorescence microscopy uses the ability of specific molecules to absorb light and then radiate it again to generate images. In the context of transfection imaging, the fluorescence channel shows which cells have successfully absorbed the transfected DNA, as only these cells express the fluorescent protein and luminesce.

Within the framework of the techniques described herein, the fluorescence channel is used to create the annotations for training the cell wall ML algorithm. Various examples are based on the finding that it is irrelevant for successfully training the cell wall ML algorithm that only some cells express the fluorescent protein and appear fluorescent in the fluorescence channel. Nevertheless it is possible to train the cell wall ML algorithm in such a way that it reliably recognizes cell walls. Details in this regard are described in detail below using the figures.

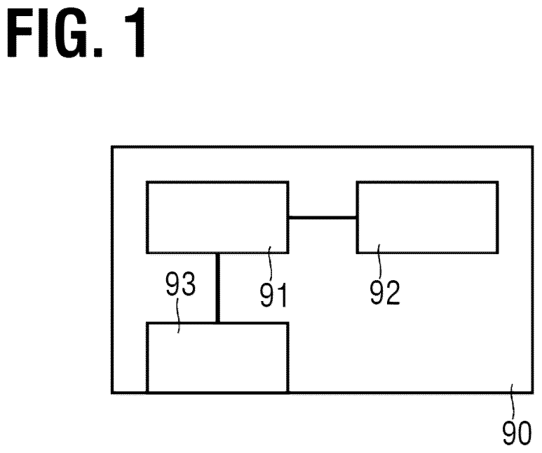

FIG. 1 shows a data processing device 90 according to various examples. The data processing device 90 comprises a processor 91 and a memory 92 and also a communication interface 93. The processor 91 can receive microscope images or general image data via the communication interface 93. For example, the processor 91 can receive transfection image data. The processor 91 can receive the image data from a database via the communication interface 93. The processor 91 can receive the image data, for example, from a microscope, for example, based on control commands which were previously sent to the microscope and which trigger the microscope to acquire corresponding image data. The processor 91 can alternatively or additionally load image data from the memory 92. The processor 91 can load program code from the memory 92 and execute it. When the processor 91 executes such a program code, the effect is that it performs techniques as described herein. In particular, the processor 91 can perform techniques in connection with the training of one or more ML algorithms. Alternatively or additionally, the processor can also recognize cell structures based on the execution of the ML algorithm as part of the inference or solve tasks such as cell instance segmentation.

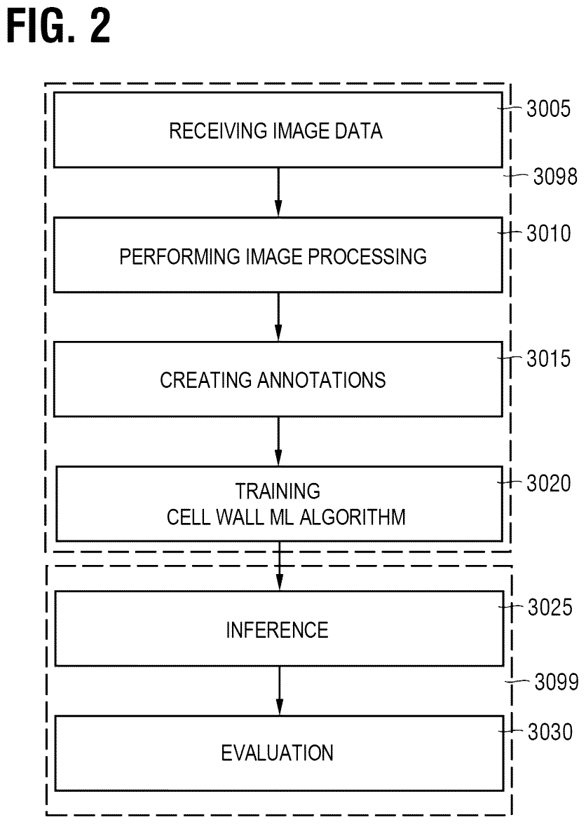

FIG. 2 is a flowchart of one exemplary method. The method of FIG. 2 can be carried out by a data processing device such as the data processing device 90 according to FIG. 1. By way of example, the method from FIG. 2 can be carried out by a processor, such as the processor 91, based on program code from a memory, such as the memory 92.

The method from FIG. 2 concerns techniques in connection with the localization of cell walls in microscope images using a cell wall ML algorithm.

The method from FIG. 2 includes both a training phase 3098 of the cell wall ML algorithm and an inference phase 3099. As a general rule, it is conceivable in the various examples described herein that either the training phase 3098 or the inference phase 3099—but not both phases—in connection are carried out.

First, aspects related to the training phase 3098 of the cell wall ML algorithm are described.

In box 3005, transfection image data are received. These include a fluorescence channel and a non-fluorescence channel. The non-fluorescence channel is typically a phase contrast channel. The transfection image data form an accumulation of cells. Not all cells express a dye. This means that not all cells in the fluorescence channel appear fluorescent. Only a fraction of all cells fluoresce in the fluorescence channel.

In box 3010, automated image processing of the fluorescence channel is then performed. This localizes boundary regions between fluorescence regions and non-fluorescence regions. Such boundary regions are arranged where cell walls are located-but not every cell wall has a boundary region: there are cells in the fluorescence channel that contain the fluorescent dye (successful transfection), as well as cells that do not (failed transfection). Nevertheless, based on this image processing, it is possible to create in box 3015 one or more types of annotations that are valuable for the subsequent training of the cell wall ML algorithm. In particular, the annotations can be determined automatically from the transfection image data.

The following text explains which types of annotations can be determined in box 3015.

A first type are cell wall annotations along localized boundary regions between fluorescence regions and non-fluorescence regions. The cell wall annotations thus mark regions where a cell wall—i.e. the edge of the cell, the cell edge—is located.

A second type are non-cell wall annotations. These non-cell wall annotations mark image regions for which it can be assumed with a sufficiently high degree of certainty that no cell wall is localized there. In particular, it would be possible for the non-cell wall annotations to be created in image regions which are adjacent to the localized boundary regions. This is based on the finding that it can be assumed with a high degree of probability in the localized boundary regions that a cell wall is localized there; it can then be in turn assumed with a high degree of probability that no cell wall is localized adjacent and parallel to such a localized cell wall.

A third type are unknown annotations. These mark image regions for which no statement can be made regarding the localization of a cell wall. Unknown annotations can be created for image regions with a minimum distance from all localized boundary regions. In image regions where no fluorescence signal is detected, no statement about the presence of a cell wall or absence of a cell wall can be made due to partial transfection. Unknown annotations may include, for example, all regions of the fluorescence channel that are not provided with a cell wall annotation or a non-cell wall annotation. The unknown annotations can still be created based on an estimation of the presence of cells in the corresponding image regions, which is generated based on the non-fluorescence channel (e.g. phase contrast). For example, by means of a grayscale variance analysis or other simple image processing and/or the use of a confluence mask, a statement can be made about the probability that a cell is located in the corresponding image region. This probability can then be taken into account further when creating the unknown annotation.

In summary, several types of annotations have been described above, which can be used as ground truth for the training of the cell wall ML algorithm. The training is then carried out in box 3020. Training of U-Net or another neural network is an iterative process that usually consists of a plurality of phases. The first step is forward propagation, in which the network processes an input image (or a batch of images) (in this case the fluorescence channel) and generates an output, e.g. localized cell walls. In this phase, the weights and distortions of the network, which were originally randomly initialized, are used to calculate the output. The generated output is then compared with the annotation (cf. box 3015) to calculate the error or discrepancy between the network prediction and the annotations. This error is often referred to as a loss function and is a measure of how well the network performs the task. Once the loss has been calculated, the backward propagation begins. During this phase, the weights and distortions of the network are adapted to minimize loss. This is typically done by the process of gradient descent, in which the derivatives of the loss are calculated in relation to each weight and each distortion. The weights and distortions are then updated in the opposite direction of the gradient to reduce the loss. This process is repeated over a plurality of epochs (passes of the entire training dataset) until the network reaches acceptable performance or the loss no longer decreases significantly. In this way, the neural network learns to effectively localize cell walls in microscope images based on the annotations, which serve as a guide.

During training, image regions with the cell wall annotations and non-cell wall annotations are normally incorporated into the training; image regions with the unknown annotation (for regions that lie outside the boundaries between the fluorescence signal and the non-fluorescence signal) are ignored. “Ignoring” the regions annotated with the unknown annotation can be realized for ML algorithms by not including the prediction of the ML algorithm in the calculation of the loss (ignore label). Instead of a hard ignore label, a soft weighting for each pixel can also be realized, e.g. depending on how far it is from another annotation, e.g. how far the corresponding pixel is from a boundary between the fluorescence signal and the non-fluorescence signal.

It is conceivable in particular that large image regions are provided with the unknown annotations. In order to still enable accurate training, a so-called consistency loss function can be calculated; in particular—but not necessarily exclusively—in image regions that are provided with the unknown annotations. See e.g. German patent application 10 2022 114 888 of Jun. 14, 2022. The consistency loss function provides a loss signal based solely on the consistency transformed inputs. Such a consistency loss function can be combined with a conventional loss function, for example in a weighted combination. However, it would also be conceivable that the consistency loss function for the entire input image is determined. In a further variant—similar to the consistency loss function—a loss signal can be generated, which minimizes the entropy of the output in the image regions with the unknown annotations, thereby maximizing the certainty of the prediction. See, for example, Wang, Dequan, et al. “Tent: Fully test-time adaptation by entropy minimization.” arXiv preprint arXiv: 2006.10726 (2020).

The training of the cell wall ML algorithm can be carried out iteratively in examples, what is known as bootstrapping. In the first iteration of the training, the cell wall ML algorithm provides a localization of cell walls, especially within a cell conglomerate. This information can then be used in a next iteration to extend the localization of edge regions of the cells even within a cell conglomerate. The training procedure is thus optionally carried out several times, with the respective result enriching the ground truth in relation to the previous iteration. In the first iteration, only the annotations that are determined based on transfection image data as described above are available. The thus trained ML algorithm then also provides cell edge predictions, which were not yet available in the training data of the first iteration (e.g. cell edges within the cell conglomerates). The prediction of the ML algorithm can now be used to generate extended annotations that are more extensive than the original annotations (but not yet perfect). This will then train the ML algorithm again, e.g. until convergence is determined.

After training, the trained cell wall ML algorithm can be applied in box 3025 to microscope images, such as a fluorescence channel. The cell wall ML algorithm can then correctly differentiate between “cell wall” and “non-cell wall” for all pixels of the microscope image—even though only partially transfected cell cultures were used for training. It was found that the cell wall ML algorithm trained in this way reliably recognizes all the cell walls in microscope images; even if the cell walls are only partially provided with corresponding cell wall annotations during training.

In a subsequent evaluation step—box 3030, generally optional—an instance segmentation of individual cells in the corresponding image data can then be performed, for example, based on the localized cell walls from box 3025. Such a determination of an instance segmentation based on localized cell walls can be implemented by means of various image processing techniques. For example, conventional image processing techniques such as watershed, flood fill, graph partitioning, etc. can be used. Here, image regions are isolated based on the (binarized) cell walls. Since the related regions thus identified can represent either individual instances or background regions, post-processing is necessary. In this process, regions that do not meet criteria for a cell instance are discarded. Different criteria are conceivable here, such as size, shape or arrangement of the region; and/or statistics/properties of phase contrast and/or fluorescence contrast in the region; and/or output of an existing algorithm for estimating cell centers and/or cell confluence, see for example European patent application 21 200 420.4 of Oct. 1, 2021. For each determined region, a check is performed as to whether it is assigned a (exactly one) cell center and/or whether the region coincides with the determined confluence.

In a further variant, an extension of the partitioning method from the German patent application 10 2021 125 575.9 of Oct. 1, 2021 can be used in box 3030. The method described therein uses a cell confluence estimation together with determined cell centers to carry out a Voronoi partitioning and thus a separation of the cell regions. Based on the determined cell walls, the method can be extended to a “weighted” partitioning, that is, Voronoi partitions do not grow equally in all directions up to coincidence, but are decelerated/accelerated locally by the determined probability of a cell wall. A partition therefore spreads more slowly when it meets a cell wall. The separation of the regions can thus also be determined more precisely for irregular shapes.

In a further variant, an ML algorithm which maps cell wall images (as output of the cell wall ML algorithm) to cell instances can be used in box 3030. The ML algorithm can be used as a model for semantic segmentation (input: cell wall image; output: segmentation mask) or as an instance segmentation model (input: cell wall image; output: individual coordinates and associated instance segmentation masks). The advantage here is that training data for this ML algorithm can be generated simply by simulation, since no textual properties of cells serve as input, but only morphological properties (shape, size, extent, arrangement of cell walls). Such input data with the associated output masks can easily be generated in large quantities by a parameterizable simulation model without manual annotations and without recordings on a device.

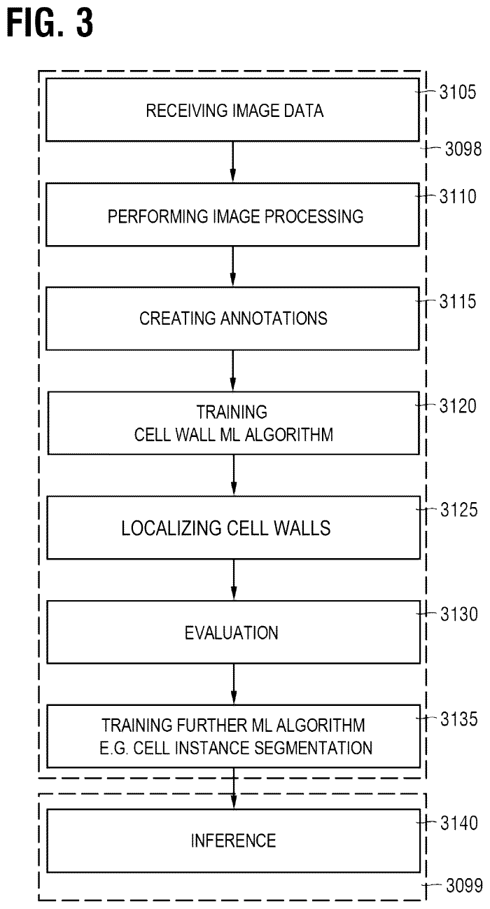

In association with FIG. 2, a variant was described above, in which, as part of the training in box 3098, a machine-learned algorithm is trained (box 3020) which localizes cell walls based on a non-fluorescence channel. In the context of the inference in box 3099, cell walls can then be localized and subsequently a further evaluation 3030 can be carried out in order to obtain, for example, a cell instance segmentation. In this variant according to FIG. 2, no ML algorithm is used that directly performs cell instance segmentation based on non-fluorescence channels. Instead, a two-step process takes place in which (i) cell walls are localized and (ii) a further evaluation, e.g. for cell instance segmentation, is carried out. Another variant in which a corresponding end-to-end ML algorithm is trained to predict cell instance segmentation as part of the training in box 3098 is shown in connection with FIG. 3.

FIG. 3 is a flowchart of one exemplary method. For example, the method of FIG. 3 can be carried out by a data processing device, such as the data processing device 90 from FIG. 1. The method from FIG. 3 can be performed, for example, by a processor—such as the processor 91—based on program code from a memory, such as the memory 92.

In box 3105, transfection image data are received. Box 3105 corresponds to box 3005 from FIG. 2.

In box 3110, image processing of the transfection image data from box 3105 is performed. Box 3110 corresponds to box 3010 from FIG. 2.

Then, one or more types of annotations are created in box 3115. Box 3115 corresponds to box 3015 from FIG. 2.

In box 3120, a cell wall ML algorithm is then trained based on the annotations from box 3115. Box 3120 corresponds to box 3020.

The cell wall ML algorithm trained in box 3120 is then used in box 3125 to localize cell walls based on non-fluorescence channels of the transfection image data from box 3105 or based on a non-fluorescence channel of further image data (for these further image data no fluorescence channel needs to be available at all).

In box 3130, an evaluation is then performed based on the cell walls found. In particular, an instance segmentation of individual cells is performed in the non-fluorescence channel.

This means that box 3125 corresponds to box 3025, and box 3130 corresponds to box 3030.

The instance segmentation in box 3130 is then used in box 3135 to train a further ML algorithm (cell instance segmentation ML algorithm) for instance segmentation. The instance segmentation from box 3130 serves as the ground truth for this training, and the non-fluorescence channel, based on which the cell walls are localized in box 3125, serves as input. Then, in the context of inference—box 3099—the cell instance segmentation ML algorithm can be used to directly segment cell walls for non-fluorescence channels, box 3140. Thus, the cell instance segmentation ML algorithm can be applied, for example, directly to conventional phase contrast microscope images and directly output a cell instance segmentation.

For example, the cell instance segmentation ML algorithm can be a U-net. The main components of the U-Net are an encoder side and a decoder side. The encoder side consists of a sequential sequence of convolutional layer blocks followed by a downsampling operation such as max pooling to reduce the spatial dimension. These layers are used to acquire contextual information and extract features of the input image. The decoder side consists of a sequential sequence of convolutional layer blocks, followed by an upsampling operation such as bilinear upsampling or transposed convolution to restore the spatial dimension. The decoder side aims to enlarge and refine the features to generate a detailed segmentation map. To connect information from the encoder side to the corresponding layer in the decoder side, the U-Net uses a skip connection. This connection bridges the spatial distance between the layers and allows the decoder side to obtain more detailed information from the encoder side for more precise segmentation results. In addition, each convolutional layer block of the U-Net uses both convolution operations and activation functions such as the ReLU function to map nonlinearities and improve the model's ability to extract features.

A further conceivable architecture for the cell instance segmentation ML algorithm is what is known as the mask R-CNN: This is an extension of the Faster R-CNN algorithm, which has been specifically developed for instance segmentation. Mask R-CNN adds an additional branch layer to the Faster R-CNN that creates a binary mask for each region of interest (ROI). In this way, Mask R-CNN can determine not only the position and category of an object in the image, but also its exact shape by way of segmentation. See Fujita, Seiya, and Xian-Hua Han. “Cell detection and segmentation in microscopy images with improved mask R-CNN.” Proceedings of the Asian Conference on Computer Vision. 2020.

A further architecture for the cell instance segmentation ML algorithm is StarDist. See Fazeli, Elnaz, et al. “Automated cell tracking using StarDist and TrackMate.” bioRxiv (2020): 2020-09. StarDist can be considered an extension of the U-Net and is based on the idea of representing the shape and position of each instance by a set of star coordinates (hence the name “StarDist”). The StarDist architecture consists of two main parts: one part that predicts the probabilities of the cell centers (similar to U-Net), and another part that predicts the star coordinates of each cell. The star coordinates represent the shape and position of the cell in relation to its center. By combining these two parts, StarDist can individually segment each cell in a microscope image and determine its exact shape and position. A major advantage of the StarDist architecture is its ability to effectively segment overlapping and touching cells, which is a common challenge in microscope image analysis. In addition, StarDist is able to handle cells of different sizes and shapes, making it a versatile solution for instance segmentation.

By means of such a method according to FIG. 3, cell instance annotations can be created in large quantities, for example on any desired phase contrast images. The large amounts of data are also necessary for accurate training, since corresponding cell instance segmentation ML algorithms are usually very data-hungry and usually require manual annotations. However, using the method described in FIG. 3, the corresponding annotations can be created automatically and the training process can be accelerated immensely.

While a variant has been described above in which a cell instance segmentation ML algorithm is trained in box 3115 to solve an instance segmentation task, other types of ML algorithms can also be trained. For example, annotations for ML algorithms for estimating cell centers or cell confluence can be created in box 3130. Each mask itself provides the confluence from the phase contrast image, and the cell center can be determined from the center of the mask. Here, too, the manual annotation effort is reduced, as mentioned above in connection with the task of cell instance segmentation.

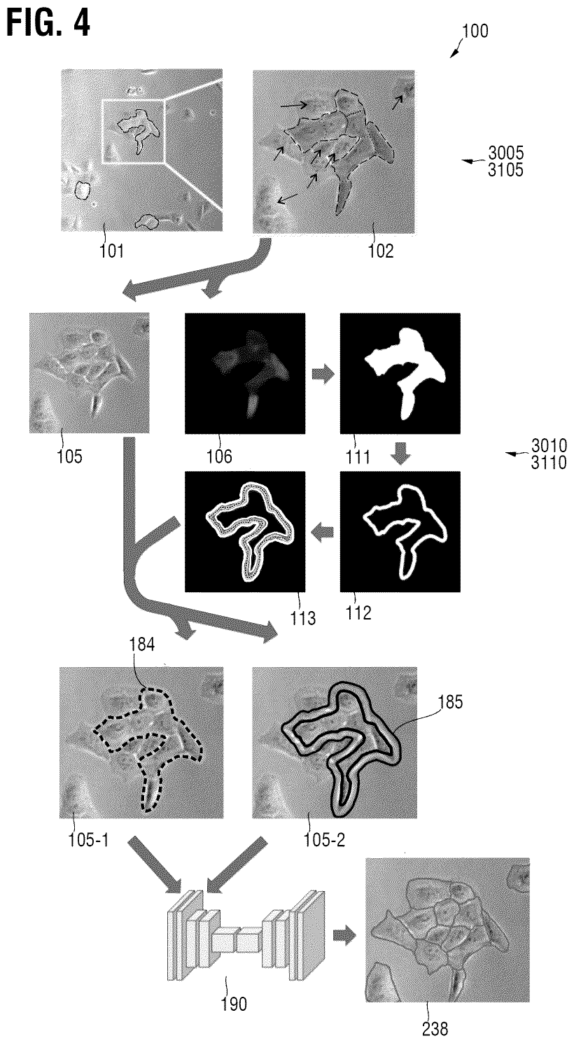

Aspects of the method from FIG. 2 and the method from FIG. 3 are illustrated in FIG. 4 based on the processing pipeline 100.

FIG. 4 illustrates a data processing pipeline 100 according to various examples. FIG. 4 shows how a cell wall ML algorithm 190 can be trained to recognize cell walls.

FIG. 4 shows, at the top, an overlay 101 of a fluorescence channel 106 with a non-fluorescence channel 105 (here: a phase contrast channel) of transfection image data (compare box 3005 and box 3105). It also shows a corresponding enlargement 102, in which it can be seen that an accumulation of cells, not all of which express a dye, is imaged into the transfection image data. (In FIG. 4, the transfected cell conglomerates are highlighted in connection using a dashed line; the contrast difference is characterized by a different color that is not easily visible in the grayscale rendering. In addition, cell boundaries within the transfected cell conglomerate are highlighted with dotted lines. Finally, non-transfected cells are marked with arrows.)

In addition, an exemplary implementation of box 3010 and box 3110 is shown, i.e. automated image processing of the fluorescence channel 106, in order to find boundary regions between fluorescence regions and non-fluorescence regions. For example, low-pass filtering of the fluorescence channel 106 can initially be performed. This type of low-pass filtering is typically used to reduce high-frequency noise in the image data, thereby improving the quality of the input image. Binarization can then be performed so that a corresponding mask image 111 is obtained. The mask image 111 can also be referred to as a binary image. There, a fixed or automatically determined threshold value is used—for example by means of Otsu's method.

In the mask image 111, contours are then recognized, for example by means of the Suzuki algorithm, see Suzuki, S. (1985). Topological structural analysis of digitized binary images by border following. Computer Vision, Graphics, and Image Processing, 30(1), 32-46. The corresponding cell wall image 112 is illustrated in FIG. 4.

The corresponding contours can then be enlarged or “washed out.” This gives a mask for the class “cell wall” (cell wall annotations). By further enlarging or further washing out of these contour edges, a mask for the class “no cell wall” is obtained (non-cell wall annotations). That is illustrated in FIG. 4 in the image 113 (where the cell wall annotations—the darker region between two bright regions—are marked with the dashed line and the non-cell wall annotations with the adjacent, bright line; see also 184, 185). As a result, two masks are obtained, one that encodes pixels that contain a cell wall; and another that encodes pixels that do not contain a cell wall. All non-encoded pixels can be assigned to an unknown annotation.

Techniques of how a cell wall annotation and a non-cell wall annotation can be created based on a transition from one pixel with a fluorescence signal to another pixel without a fluorescence signal have been described above.

It should be noted that there are also other possible implementation variants of the image processing algorithm for cell wall recognition. These can include both conventional algorithms and ML algorithms, in particular deep neural networks. In the latter case, a network that is intended to recognize the interface between the fluorescence signal and the non-fluorescence signal has a much easier task and is therefore trainable more easily and with less data than a network that is intended to recognize cell boundaries directly in phase contrast images.

Variants or extensions that utilize the spatial structure of the fluorescence channel would also be conceivable. This allows, for example, cell edges within a cell conglomerate to be recognized, even if the cells in the cell conglomerate all express the fluorescent dye. Such an analysis of the structure of the fluorescence channel can be carried out, for example, by means of a corresponding ML algorithm.

FIG. 4 also shows an overlay 105-1 of the cell wall annotations 184 (highlighted by the short dashed line) with the phase contrast channel 105. In addition, an overlay 105-2 of the non-cell wall annotations 185 (solid line) with the phase contrast channel 105 is shown. These annotations (for example, together with the unknown annotation) then serve as the ground truth for the training of the machine-learned algorithm 190, which recognizes cell walls as output 238.

The output 238 of the ML algorithm 190, i.e. the estimated cell walls, implicitly contains the information of the individual cell instances. Optionally, the estimated cell walls can then be converted explicitly into individual cell segments by post-processing. This has already been described above in conjunction with box 3030 and box 3130. The output 238 can either be used to create annotations for training a further machine-learned algorithm—for example, for direct instance segmentation of cells—compare FIG. 3; or the output 238 can be evaluated in the context of the inference to segment cells in this way (see FIG. 2).

In summary, techniques have been described above that allow the creation of annotations for the training of one or more ML algorithms based on transfection image data. The transfection image data show a multiplicity of cells, only some of which express the dye. The annotations can then be used to train an ML algorithm to localize all cell walls. Based on this, a cell instance segmentation can be performed or a further ML algorithm can be trained to solve a corresponding task directly.

The features of the embodiments and aspects of the invention described above can be combined with one another. In particular, the features can be used not only in the combinations described but also in other combinations or on their own, without departing from the scope of the invention.

Various examples have been described above in connection with the automated image processing of a fluorescence channel from transfection image data in order to thus obtain boundary regions between fluorescence regions and non-fluorescence regions. Based on this, cell wall annotations are then created along the localized boundary regions. In some variants, it would also be conceivable that the cell wall annotations are manually created based on general image data—for example, based on a phase contrast channel. Even in such an example, the training of the cell wall ML algorithm can then be performed, as described in detail above, based on these manually created cell wall annotations. Even in such a variant with the manual creation of cell wall annotations, the training effort can be significantly reduced, compared to a reference implementation in which cell instances are annotated manually. Manual annotation of any contiguous cells is easier and faster than full annotation of all individual cells in an image.

Furthermore, techniques in which cells fluoresce in a fluorescence channel have been described above. It would also be possible that only cell walls fluoresce in the fluorescence channel, i.e. the inside of the cell does not fluoresce. The corresponding algorithms described herein can be directly adapted to provide cell wall annotations based on such information.

What is claimed is:

1. A computer-implemented method, wherein the method comprises:
receiving transfection image data comprising a fluorescence channel and a non-fluorescence channel, wherein the transfection image data represent an accumulation of cells, not all of which express a dye,
performing automated image processing of the fluorescence channel to localize boundary regions between fluorescence regions and non-fluorescence regions,
creating cell wall annotations along the localized boundary regions, and
training a machine-learned algorithm based on the cell wall annotations as ground truth and the non-fluorescence channel as input.

2. The computer-implemented method of claim 1, wherein the method furthermore comprises:
creating non-cell wall annotations in image regions which are adjacent to the localized boundary regions,
wherein said training is furthermore based on the non-cell wall annotations as a further ground truth.

3. The computer-implemented method of claim 1, wherein the method furthermore comprises:
creating unknown annotations in image regions with a minimum distance from all localized boundary regions,
wherein the training is furthermore based on the unknown annotations as a further ground truth.

4. The computer-implemented method of claim 3,
wherein the unknown annotations are furthermore created based on an estimation of the presence of cells in the corresponding image regions based on the non-fluorescence channel.

5. The computer-implemented method of claim 3,
wherein, a prediction of the machine-learned algorithm in regions with the unknown annotation is not included in a loss function during the training of the machine-learned algorithm.

6. The computer-implemented method of claim 3,
wherein a prediction of the machine-learned algorithm in regions with the unknown annotation is included weighted in a loss function during the training of the machine-learned algorithm, wherein a weighting is determined in dependence on a distance from at least one other annotation.

7. The computer-implemented method of claim 1, wherein the method furthermore comprises:
applying the machine-learned algorithm for localizing cell walls in a non-fluorescence channel of inference image data.

8. The computer-implemented method of claim 7, wherein the method furthermore comprises:
based on the localized cell walls in an output of the machine-learned algorithm: performing instance localization of individual cells in the inference image data.

9. The computer-implemented method of claim 8,
wherein performing the instance localization recognizes contiguous regions and then separates each contiguous region into cell instance or background.

10. The computer-implemented method of claim 1, wherein the method furthermore comprises:
applying the machine-learned algorithm for localizing cell walls in the non-fluorescence channel of the transfection image data or other image data,
based on the cell walls found: performing instance localization of individual cells in the non-fluorescence channel, and
training a further machine-learned algorithm based on the instance localization as the ground truth and the non-fluorescence channel as input.

11. The computer-implemented method of claim 1, further comprising:
evaluating a spatial structure of fluorescent regions in the fluorescence channel,
wherein the cell wall annotations are furthermore created based on the spatial structure.

12. The computer-implemented method of claim 1,
wherein the training of the machine-learned algorithm uses a consistency loss function or a loss function which minimizes a degree of entropy of an output of the machine-learned algorithm in at least some image regions.

13. A data processing device, which is configured to:
receive transfection image data comprising a fluorescence channel and a non-fluorescence channel, wherein the transfection image data represent an accumulation of cells, not all of which express a dye,
perform automated image processing of the fluorescence channel to localize boundary regions between fluorescence regions and non-fluorescence regions,
create cell wall annotations along the localized boundary regions, and
train a machine-learned algorithm based on the cell wall annotations as ground truth and the non-fluorescence channel as input.
